(19) United States
(12) Patent Application Publication
(10) Pub. No.: US 2009/0149714 A1
(43) Pub. Date: Jun. 11, 2009

(54) SURGICAL DEVICES AND METHODS

(76) Inventor: Frank BONADIO, Bray (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/327,211

(22) Filed: Dec. 3, 2008

Related U.S. Application Data
(60) Provisional application No. 60/992,426, filed on Dec. 5, 2007.

(51) Int. Cl. A61B 1/32

(52) U.S. Cl. 600/201

(57) ABSTRACT

A retractor is used for natural orifice transluminal endoscopic surgery. The procedure may be performed via a transgastric, a transvaginal, or a transcolonic route.

This application claims the benefit of U.S. Provisional Application No. 60/992,426, filed Dec. 5, 2007.

The content of this provisional application is incorporated herein by reference.

### INTRODUCTION

This invention relates to an apparatus for inserting a surgical device at least partially through a wound opening, to a device for retracting the sides of a wound opening, and to a method of performing a surgical procedure.

### STATEMENTS OF INVENTION

The invention provides methods and devices for use in natural orifice transluminal endoscopic surgery in which a procedure is performed via a transgastric, transvaginal or transcolonic route.

According to the invention there is provided an apparatus for inserting a surgical device at least partially through a wound opening in a tissue wall between a first internal body cavity and a second internal body cavity, the apparatus comprising a conveying device insertable at least partially through the wound opening to convey the surgical device at least partially through the wound opening.

In one embodiment of the invention the apparatus is configured to extend proximally from a first internal body cavity to a location externally of the body. The apparatus may be configured to extend through a natural orifice.

The apparatus may comprise an operator handle configured to be located externally of a body.

The length of the apparatus between the proximal end of the apparatus and the distal end of the apparatus may be greater than 15 cm. The length of the apparatus may be greater than 30 cm. The length of the apparatus may be greater than 45 cm.

In one case the conveying device comprises a housing portion for receiving at least part of a surgical device to couple the surgical device to the conveying device.

In another aspect of the invention there is provided a device for retracting the sides of a wound opening in a tissue wall between a first internal body cavity and a second internal body cavity, the device comprising:

a distal anchoring member for insertion into the second internal body cavity, and

a retractor member extending proximally from the distal anchoring member to retract laterally the sides of the wound opening.

In one embodiment of the invention the device comprises a proximal member for location in a first internal body cavity. The retractor member may extend from the distal anchoring member to the proximal member. The retractor member may be configured to extend proximally of the proximal member from a first internal body cavity to a location externally of the body. The retractor member may be configured to extend through a natural orifice. The retractor member maybe configured to extend proximally of the proximal member by a distance of greater than 15 cm. The retractor member may be configured to extend proximally of the proximal member by a distance of greater than 30 cm. The retractor member may be configured to extend proximally of the proximal member by a distance of greater than 45 cm.

In one case the proximal member is movable relative to the distal anchoring member to retract laterally the sides of a wound opening. The device may comprise an actuator member to move the proximal member relative to the distal anchoring member. The actuator member may be configured to extend proximally from a first internal body cavity to a location externally of the body. The actuator member may be configured to extend through a natural orifice. The length of the actuator member between the proximal end of the actuator member and the distal end of the actuator member may be greater than 15 cm. The length of the actuator member may be greater than 30 cm. The length of the actuator member may be greater than 45 cm.

According to another aspect of the invention there is provided a method of performing a surgical procedure, the method comprising the steps of:

creating a wound opening in a tissue wall between a first internal body cavity and a second internal body cavity, and

retracting laterally the sides of the wound opening.

In one embodiment of the invention the method comprises the step of inserting a surgical device at least partially through the wound opening. The wound opening may be retracted using the surgical device. The surgical device may be delivered to the first internal body cavity through a natural orifice.

In one case the method comprises the step of inserting a surgical instrument from the first internal body cavity through the wound opening into the second internal body cavity. The method may comprise the step of performing a surgical procedure in the second internal body cavity. The method may comprise the step of withdrawing one or more body parts from the second internal body cavity through the wound opening into the first internal body cavity.

### BRIEF DESCRIPTION OF THE DRAWINGS

The invention will be more clearly understood from the following description of an embodiment thereof, given by way of example only, with reference to the accompanying drawings, in which:

FIGS. 1 to 4 are partially cross-sectional side views illustrating insertion of a surgical device at least partially through a wound opening;

FIGS. 5 and 6 are partially cross-sectional side views illustrating retraction of the sides of the wound opening;

FIG. 7 is a partially cross-sectional side view illustrating insertion of a surgical instrument through the wound opening; and

FIGS. 8 to 13 are views illustrating accessing the abdominal cavity through the inner wall of the vagina.

### DETAILED DESCRIPTION

Referring to the drawings there is illustrated a device 1 according to the invention for retracting the sides of a wound opening 2, and an apparatus 3 according to the invention for inserting the device 1 at least partially through the wound opening 2. The wound opening 2 is provided in a tissue wall 13 between a first internal body cavity and a second internal body cavity. The first internal body cavity may be the stomach 12, and the second internal body cavity may be the abdominal cavity 14. Alternatively the first internal body cavity may be the vagina.

This invention is suitable for use with natural orifice transluminal endoscopic surgery. In this case the oesophagus 11/stomach 12 is the natural orifice. The concept is to pierce through the stomach wall 13 into the abdominal cavity 14 and keep the incision 2 open with the device 1, which also allows the abdominal cavity 14 to be insufflated. A scope 16 with multiple instrument working channels may be passed through the device 1 to perform abdominal surgery.

The device 1 comprises a distal anchoring ring 4 for insertion into the abdominal cavity 14, a proximal ring assembly 5 for location in the stomach 12, and a retracting sleeve 6 to retract laterally the sides of the wound opening 2. One end of the sleeve 6 is fixed to the proximal ring assembly 5. The sleeve 6 extends distally from the proximal ring assembly 5 to the distal anchoring ring 4, is looped around the distal anchoring ring 4, and extends proximally to the proximal ring assembly 5. The sleeve 6 extends through the proximal ring assembly 5 and extends proximally of the proximal ring assembly 5.

The sleeve 6 may extend proximally of the proximal ring assembly 5 by a distance of greater than 15 cm, preferably greater than 30 cm, most preferably greater than 45 cm. This enables the sleeve 6 to extend proximally from the stomach 12 through the mouth 10 to a location externally of the body.

The apparatus 3 comprises a conveying device 9 which is insertable at least partially through the wound opening 2 to convey the device I at least partially through the wound opening 2.

The conveying device 9 comprises an operator handle 7 at the proximal end of the conveying device 9, and a housing portion 8 at the distal end of the conveying device 9. The distal anchoring ring 4 of the device 1 is received in the housing portion 8 to couple the device 1 to the conveying device 9.

The conveying device 9 comprises a blunt, bladeless tip 10 to create the wound opening 2 by forcing tissue apart.

The length of the conveying device 9 between the proximal end of the conveying device 9 and the distal end of the conveying device 9 is greater than 15 cm, preferably greater than 30 cm, most preferably greater than 45 cm. This enables the conveying device 9 to extend proximally from the stomach 12 through the mouth 10 to a location externally of the body, with the operator handle 7 located externally of the body.

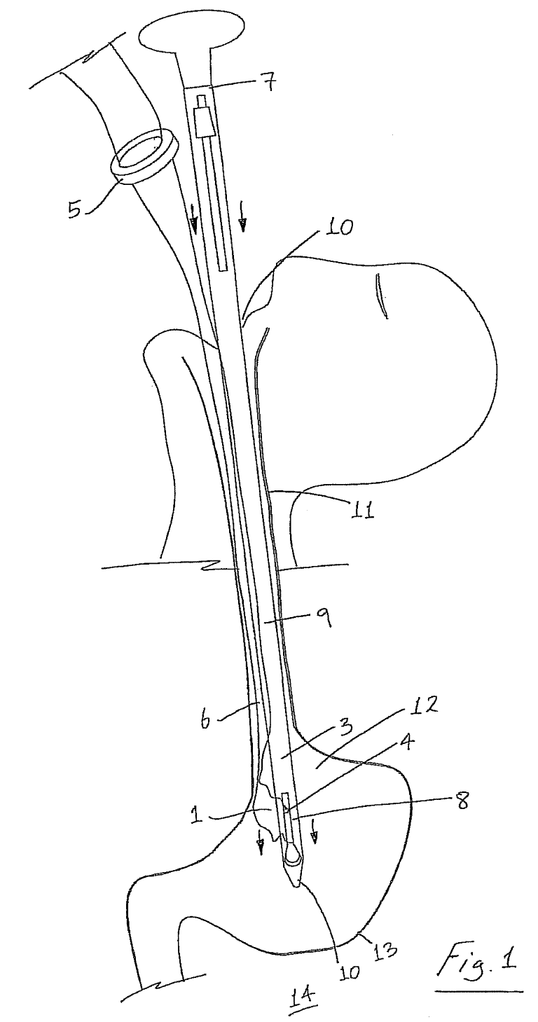

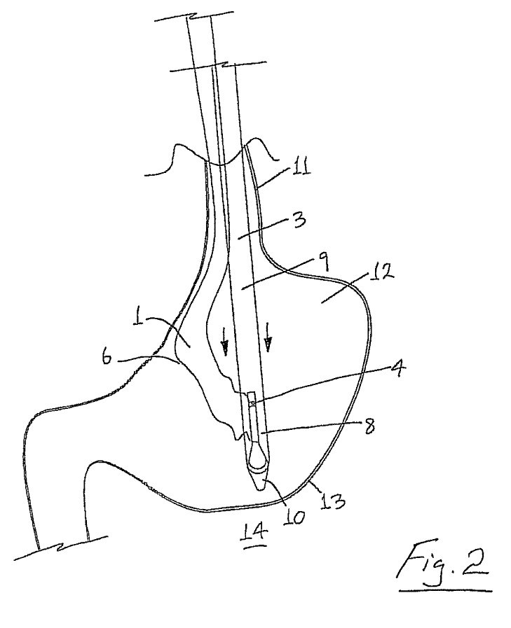

In use the distal anchoring ring 4 of the device 1 is inserted into the housing portion 8 of the conveying device 9. The conveying device 9 is inserted through the mouth 10 of a patient, through the oesophagus 11 and into the stomach 12 (FIG. 1). The conveying device 9 is advanced distally towards the tissue wall 13 of the stomach 12 (FIG. 2), and is forced through the tissue wall 13 to create the wound opening 2 by forcing tissue apart (FIG. 3).

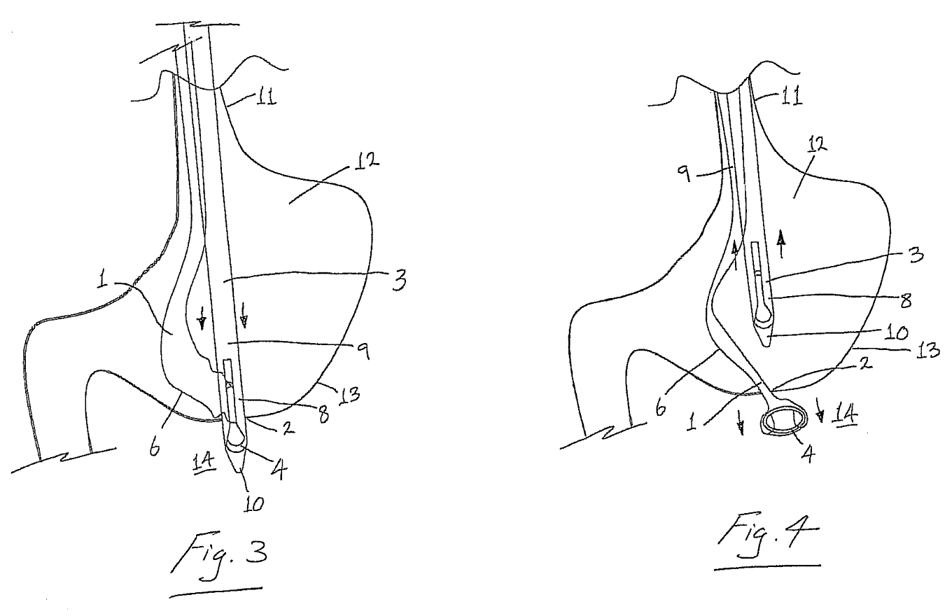

The distal anchoring ring 4 of the device 1 is ejected from the housing portion 8 into the abdominal cavity 14 (FIG. 4), and the conveying device 9 is withdrawn from the stomach 12 through the oesophagus 11 and out of the mouth 10.

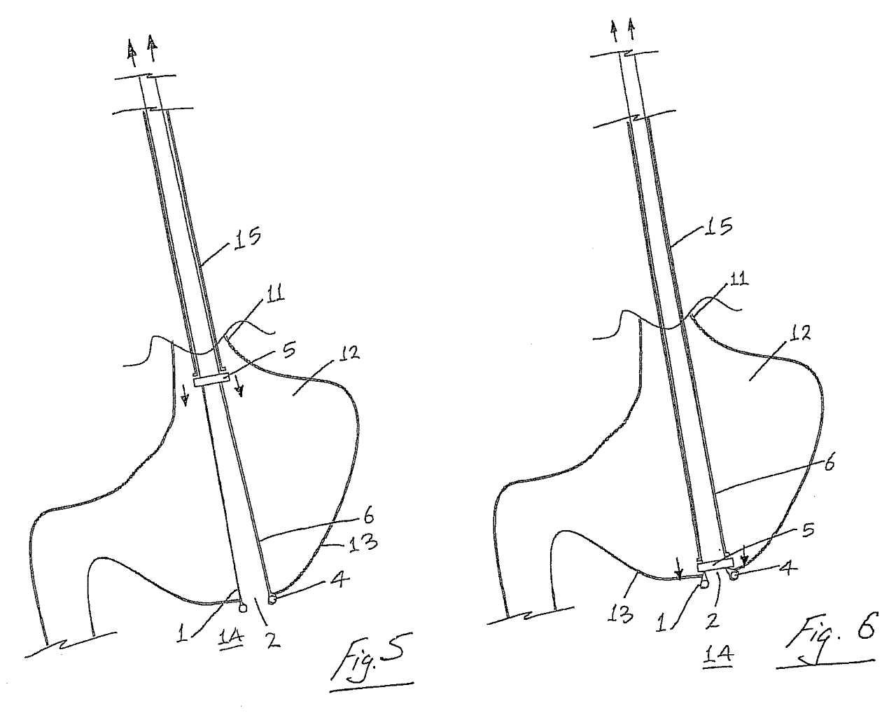

The proximal ring assembly 5 is moved distally relative to the distal anchoring member 4 using an actuator pusher 15, while the sleeve 6 is pulled taut (FIG. 5). The length of the actuator pusher 15 between the proximal end of the actuator pusher 15 and the distal end of the actuator pusher 15 is greater than 15 cm, preferably greater than 30 cm, most preferably greater than 45 cm. This enables the actuator pusher 15 to extend proximally from the stomach 12 through the mouth 10 to a location externally of the body. When the proximal ring assembly 5 reaches the tissue wall 13, the wound opening 2 is retracted fully (FIG. 6). The actuator pusher 15 is then withdrawn from the stomach 12 through the oesophagus 11 and out of the mouth 10.

One or more surgical instruments 16 may be inserted through the mouth 10, through the oesophagus 11, into the stomach 12, through the retracted wound opening 2 and into the abdominal cavity 14. The surgical instruments 16 may perform surgical procedures in the abdominal cavity 14, and may withdraw one or more body parts from the abdominal cavity 14 through the retracted wound opening 2, through the stomach 12, through the oesophagus 11 and out of the mouth 10.

FIG. 1 illustrates the trans-gastric abdominal access system which creates a working channel through the stomach 12. FIG. 1 illustrates the distal ring 4, the long sleeve 6, the outer proximal ring 5 which may include a gel valve, the long injector introducer 9, the stomach 12, and the patient's head.

FIG. 3 illustrates the insert injector introducer tip 10 through the stomach wall 13 and into the abdominal cavity 14.

FIG. 4 illustrates ejecting the distal ring 4. FIG. 5 illustrates the outer proximal ring 5, the overtube 15, with tension applied to the sleeve 6 and advancing the outer proximal ring 5 with the overtube 15. FIG. 6 illustrates full retraction.

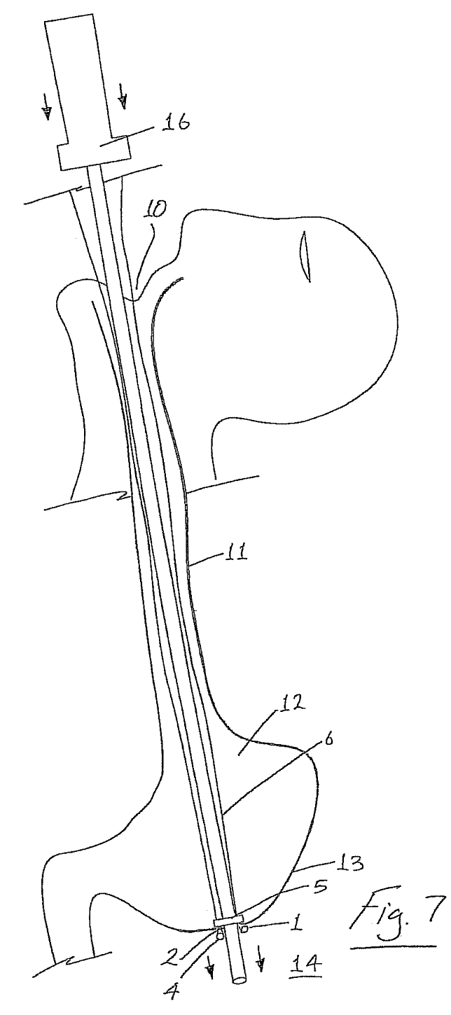

FIG. 7 illustrates the scope 16 which has working channels for instruments to pass through.

Referring to FIGS. 8 to 13 there is illustrated the use of a system 100 to facilitate access into the abdominal cavity 101 through the inner wall 102 of the vagina 104. The system is similar to that described alone and like parts use assigned the same reference numerals.

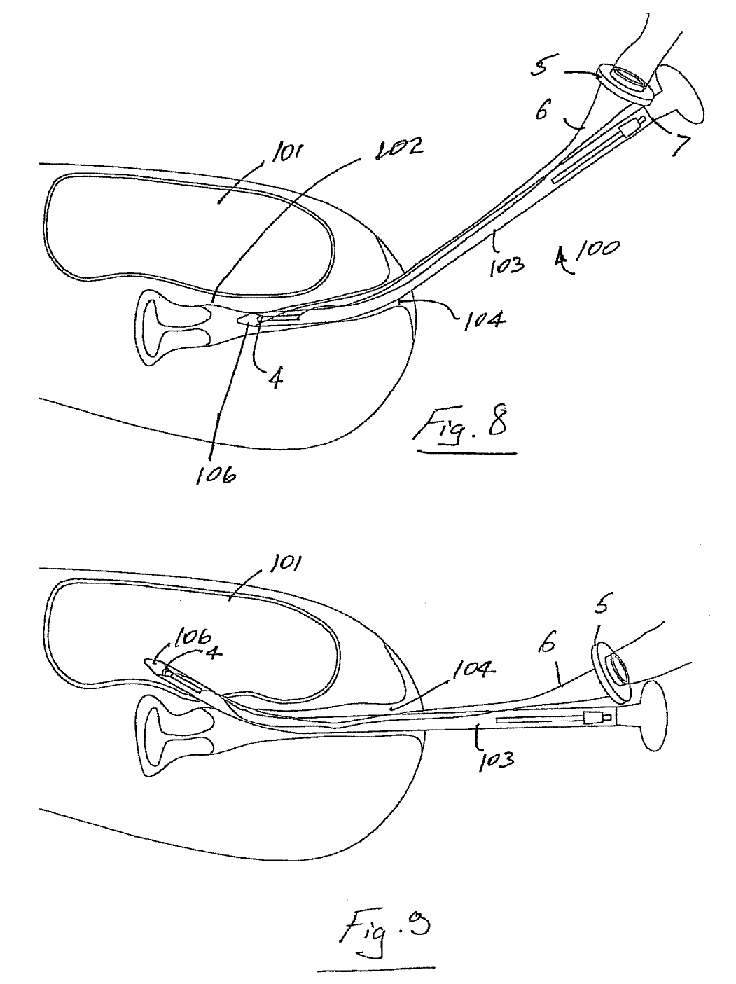

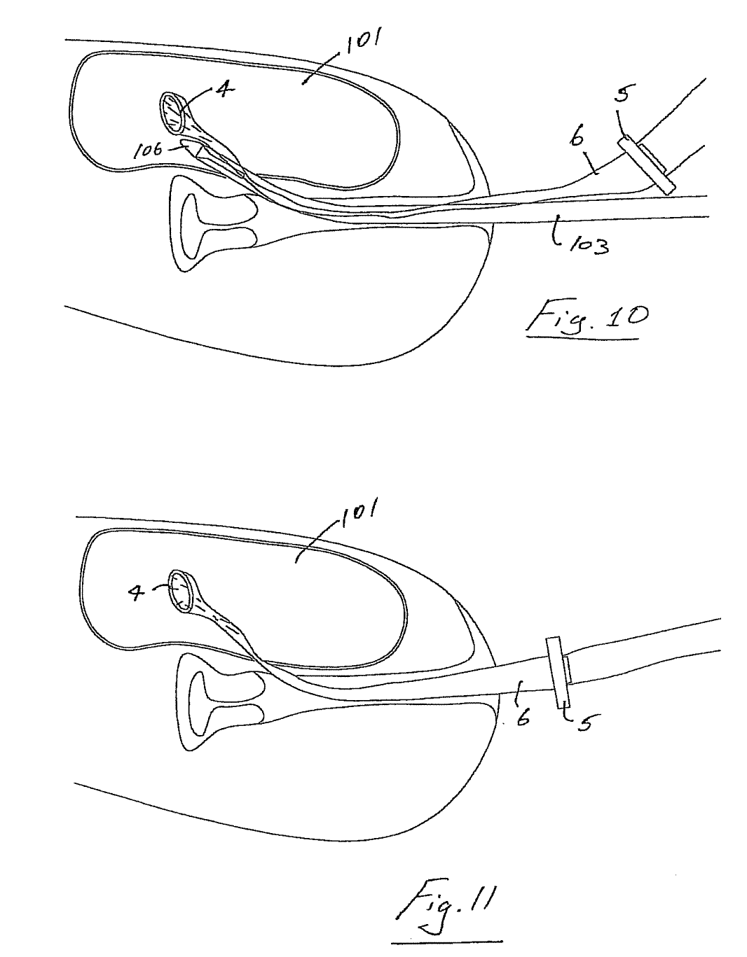

In FIG. 8 a curved introducer 103 is used to deliver the device through the vagina 104. The distal tip 106 of the introducer 103 is illustrated in FIG. 9 having being passed through the inner vaginal wall 102 and into the abdominal cavity. The distal ring is then ejected from the introducer 103 and into the abdominal cavity 101 (FIG. 10). The introducer 103 is then removed leaving the distal ring and attached sleeve in situ [FIG. 11].

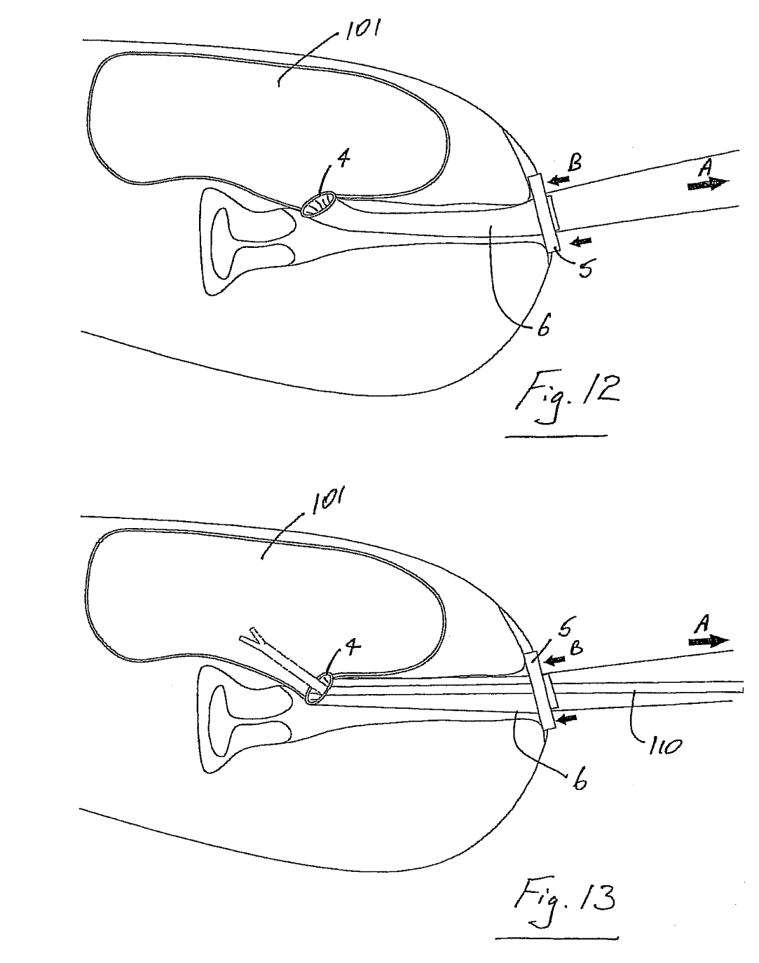

Referring to FIG. 12, the sleeve is pulled towards the surgeon in the direction of the arrow A and the outer proximal ring is pushed in the direction of the arrow B, the distal ring is drawn towards the opening into the abdominal cavity and the incision is retracted. The system provides a pathway for the surgeon from the outside the patient into the abdominal cavity for an instrument 110 such as a grasper, clip applier, and/or endoscope to carry out any described procedure [FIG. 13]. The procedure is relatively easy to carry out and minimises patient trauma and the time required for recovery and wound healing.

The invention is not limited to the embodiments hereinbefore described, with reference to the accompanying drawings, which may be varied in construction and detail.

1. An apparatus for inserting a surgical device at least partially through a wound opening in a tissue wall between a first internal body cavity and a second internal body cavity, the apparatus comprising a conveying device insertable at least partially through the wound opening to convey the surgical device at least partially through the wound opening.

2. An apparatus as claimed in claim 1 wherein the apparatus is configured to extend proximally from a first internal body cavity to a location externally of the body.

3. An apparatus as claimed in claim 2 wherein the apparatus is configured to extend through a natural orifice.

4. An apparatus as claimed in claim 2 wherein the apparatus comprises an operator handle configured to be located externally of a body.

5. An apparatus as claimed in claim 1 wherein the length of the apparatus between the proximal end of the apparatus and the distal end of the apparatus is greater than 15 cm.

6. An apparatus as claimed in claim 5 wherein the length of the apparatus is greater than 30 cm.

7. An apparatus as claimed in claim 6 wherein the length of the apparatus is greater than 45 cm.

8. An apparatus as claimed in claim 1 wherein the conveying device comprises a housing portion for receiving at least part of a surgical device to couple the surgical device to the conveying device.

9. A device for retracting the sides of a wound opening in a tissue wall between a first internal body cavity and a second internal body cavity, the device comprising:
a distal anchoring member for insertion into the second internal body cavity, and
a retractor member extending proximally from the distal anchoring member to retract laterally the sides of the wound opening.

10. A device as claimed in claim 9 wherein the device comprises a proximal member for location in a first internal body cavity.

11. A device as claimed in claim 10 wherein the retractor member extends from the distal anchoring member to the proximal member.

12. A device as claimed in claim 10 wherein the retractor member is configured to extend proximally of the proximal member from a first internal body cavity to a location externally of the body.

13. A device as claimed in claim 12 wherein the retractor member is configured to extend through a natural orifice.

14. A device as claimed in claim 10 wherein the retractor member is configured to extend proximally of the proximal member by a distance of greater than 15 cm.

15. A device as claimed in claim 14 wherein the retractor member is configured to extend proximally of the proximal member by a distance of greater than 30 cm.

16. A device as claimed in claim 15 wherein the retractor member is configured to extend proximally of the proximal member by a distance of greater than 45 cm.

17. A device as claimed in claim 10 wherein the proximal member is movable relative to the distal anchoring member to retract laterally the sides of a wound opening.

18. A device as claimed in claim 17 wherein the device comprises an actuator member to move the proximal member relative to the distal anchoring member.

19. A device as claimed in claim 18 wherein the actuator member is configured to extend proximally from a first internal body cavity to a location externally of the body.

20. A device as claimed in claim 19 wherein the actuator member is configured to extend through a natural orifice.

21. A device as claimed in claim 18 wherein the length of the actuator member between the proximal end of the actuator member and the distal end of the actuator member is greater than 15 cm.

22. A device as claimed in claim 21 wherein the length of the actuator member is greater than 30 cm.

23. A device as claimed in claim 22 wherein the length of the actuator member is greater than 45 cm.

24. A method of performing a surgical procedure, the method comprising the steps of:
creating a wound opening in a tissue wall between a first internal body cavity and a second internal body cavity, and
retracting laterally the sides of the wound opening.

25. A method as claimed in claim 24 wherein the method comprises the step of inserting a surgical device at least partially through the wound opening.

26. A method as claimed in claim 25 wherein the wound opening is retracted using the surgical device.

27. A method as claimed in claim 25 wherein the surgical device is delivered to the first internal body cavity through a natural orifice.

28. A method as claimed in any of claims 24 wherein the method comprises the step of inserting a surgical instrument from the first internal body cavity through the wound opening into the second internal body cavity.

29. A method as claimed in any of claims 24 wherein the method comprises the step of performing a surgical procedure in the second internal body cavity.

30. A method as claimed in any of claims 24 wherein the method comprises the step of withdrawing one or more body parts from the second internal body cavity through the wound opening into the first internal body cavity.
